(19) United States
(12) Patent Application Publication
(10) Pub. No.: US 2019/0374948 A1
(43) Pub. Date: Dec. 12, 2019

(54) ANTIMICROBIAL SUSCEPTIBILITY TEST KITS

(71) Applicant: Technion Research & Development Foundation Limited, Haifa (IL)

(72) Inventors: Jonathan AVESAR, San Diego, CA (US); Shulamit LEVENBERG, Moreshet (IL); Dekel ROSENFELD, Kiryat Bialik (IL); Yaron Joseph BLINDER, Moshav Shoresh (IL)

(73) Assignee: Technion Research & Development Foundation Limited, Haifa (IL)

(21) Appl. No.: 16/486,879

(22) PCT Filed: Feb. 23, 2018
(86) PCT No.: PCT/IL2018/050082

§ 371 Date: Aug. 19, 2019

Related U.S. Application Data
(60) Provisional application No. 62/460,869, filed on Feb. 19, 2017.

(51) Int. Cl. B01L 3/00; C12Q 1/18

(52) U.S. Cl. CPC B01L 3/502723; C12Q 1/18; B01L 2200/0684; B01L 2200/16; B01L 2400/084; B01L 2300/0877; B01L 2200/061; B01L 2200/0636

(57) ABSTRACT

A microfluidic device may include a microstructure formed in a substrate, the microstructure including a primary channel with a first end and a second end, and a plurality of chambers that open to the primary channel. At least two openings coupled to the first end of the primary channel may be used to load at least two fluid streams into the device through the first end of the primary channel to flow along the primary channel from the first end to the second end into the plurality of chambers, each chamber of the plurality of chambers having a volume less than 100 nanoliters and connected by a vent to a secondary channel in the micro structure, a width of the vent configured to enable a gas to escape from the chamber to the secondary channel while inhibiting the flow of said at least first and second fluid streams into the secondary channel.

### FIELD OF THE INVENTION

The present invention relates to antimicrobial susceptibility testing and particularly to antimicrobial susceptibility test kits.

### BACKGROUND OF THE INVENTION

Antibiotic and antimicrobial resistance (AMR) is a major global health concern. Due to an overuse of antibiotics, certain bacteria may develop AMR, which may reduce the ability of antibiotics to kill the pathogens and the efficiency of antibiotics to be used in alleviating many diseases resulting from bacteria with AMR. AMR has resulted in prolonged sicknesses of millions of people annually and costs billions of dollars in the U.S. annually in increased healthcare costs.

Antimicrobial susceptibility testing (AST) may be used to probe for resistant phenotypes of pathogens and to determine a minimal dosage or minimal inhibitory concentration (MIC) of an antibiotic needed to inhibit the pathogen. However, routine clinical tests for probing resistant or non-resistant pathogens in a subject may typically need two days to one week before receiving the results of AST from the time that the samples were collected. For example, the collected sample may need 24-48 hours of incubation before AST may be initiated. In the case of bacteremia and sepsis, a blood culture step may be needed with five days of incubation. Antimicrobial susceptibility testing may then take an additional 8-24 hours.

When a subject, such as a human subject, is sick, and a health care professional, such as a doctor, may suspect that the subject has an infection, the doctor may initiate antimicrobial susceptibility testing to identify susceptible/resistant phenotypes. However, before the results of antimicrobial susceptibility testing may be available, the doctor, in parallel, may prescribe an antibiotic for the subject in order to prevent a worsening of the condition due to the long time to receive the AST results. The antibiotic may be administered in large doses with a broad spectrum of activity to ensure its efficacy on the target pathogen. However, this very approach may facilitate the emergence of AMR in a clinic and may damage microbiota in the subject.

Thus, it may be desirable to have low cost and rapid AST kits for quickly and efficiently identifying one or a plurality of antibiotics to kill pathogens making the subject sick, and a variety of metrics related to the identified one or a plurality of antibiotics to assist the health care professional. Low cost, rapid antimicrobial susceptibility test kits may prevent the need for administering large unnecessary doses of the broad spectrum antibiotics to the subject and to reduce the emergence of AMR.

### SUMMARY OF THE INVENTION

There is thus provided, in accordance with some embodiments of the present invention, a microfluidic device which may include a microstructure formed in a substrate. The microstructure may include a primary channel with a first end and a second end, and a plurality of chambers that open to the primary channel. At least two openings may be coupled to the first end of the primary channel, to load at least two fluid streams into the device through the first end of the primary channel to flow along the primary channel from the first end to the second end into the plurality of chambers, each chamber of the plurality of chambers having a volume less than 100 nanoliters and may be connected by a vent to a secondary channel in the microstructure, a width of the vent being configured to enable a gas to escape from the chamber to the secondary channel while inhibiting the flow of said at least first and second fluid streams into the secondary channel. One or a plurality of retaining channels may be coupled between the primary channel and the secondary channel to allow a retaining fluid in the primary channel to flow into the secondary channel while inhibiting the flow of fluid of said at least two fluid streams into the secondary channel.

In accordance with some embodiments of the present invention, the microfluidic device may include a second end opening coupled to the second end of the primary channel, and wherein the retaining fluid may be loaded into the primary channel.

In accordance with some embodiments of the present invention, the plurality of chambers that open to the primary channel may be arranged in a first array of chambers from the plurality of chambers positioned along a first side of the primary channel and a second and a second array of chambers from the plurality of chambers positioned along a second side of the primary channel substantially opposite to the first array.

In accordance with some embodiments of the present invention, the vent may include one or a plurality of slits.

In accordance with some embodiments of the present invention, an opening between a chamber of the plurality of chambers and the primary channel includes narrowing structure.

There is further provided, in accordance with some embodiments of the present invention, an antimicrobial susceptibility test (AST) kit which may include a microstructure formed in a substrate. The microstructure may include a primary channel with a first end and a second end, and a plurality of chambers open to the primary channel, each chamber in the plurality of chambers having a volume less than 100 nanoliters and may be connected by a vent to a secondary channel in the microstructure, a width of the vent may be configured to enable a gas to escape from the chamber to the secondary channel while inhibiting the flow of a sample fluid into the secondary channel, where each chamber in the plurality of chambers includes an antibiotic with a concentration of the antibiotic dependent on a position of the chamber of said plurality of chambers along the primary channel. At least one first end opening may be coupled to the first end of the primary channel and a second end opening may be coupled to the second end of the primary channel to enable a sample fluid to be loaded into the device either through the at least one first end opening or the second end opening, to flow along the primary channel into the plurality of chambers, and to mix with the antibiotic in each chamber. A retaining channel may be coupled between the primary channel and the secondary channel which may allow a retaining fluid in the primary channel to flow into the secondary channel while inhibiting the flow of the sample fluid into the secondary channel so as to isolate droplets of the sample fluid in each chamber of the plurality of chambers.

In accordance with some embodiments of the present invention, the sample fluid may include a bacterial sample solution.

In accordance with some embodiments of the present invention, the antibiotic may include an antibiotic fluid.

In accordance with some embodiments of the present invention, the antibiotic may include a lyophilized antibiotic solute, and the mass of the lyophilized antibiotic solute is related to the concentration of the antibiotic solution prior to lyophilization.

In accordance with some embodiments of the present invention, the test kit may include at least two microstructures on the substrate and a common opening to simultaneously load the sample fluid into the primary channel of the at least two microstructures.

In accordance with some embodiments of the present invention, the retaining fluid may include air or FC-40 oil.

There is further provided, in accordance with some embodiments of the present invention, a method for forming droplets with gradually varied concentrations in a microfluidic device including in a microstructure formed in a substrate, the microstructure including a primary channel with a first end and a second end, and a plurality of chambers that open to the primary channel. The method may include loading through at least two first end openings coupled to the first end of the primary channel, concurrently, at least two fluid streams into the primary channel, which may form, when the at least two fluid streams mix, a fluid mixture having a concentration gradient along the primary channel and the plurality of chambers that are open to that primary channel. Upon loading the plurality of chambers with the fluid mixture, a retaining fluid may be introduced into the primary channel to purge the fluid mixture from the primary channel while retaining droplets of the fluid mixture in the plurality of chambers—a droplet of said droplets in each of the plurality of chambers, so as to exhibit gradually varied concentrations in the droplets in the plurality of chambers along the primary channel.

In accordance with some embodiments of the present invention, the retaining fluid may include a shearing fluid introduced into the first end of the primary channel through a purge opening coupled to the first end so as to purge the fluid of said at least two fluid streams from the primary channel.

In accordance with some embodiments of the present invention, the shearing fluid may include air or oil.

In accordance with some embodiments of the present invention, the method may include computing the concentration of the solute in the droplet using a two-dimensional advection-diffusion equation.

In accordance with some embodiments of the present invention, loading the at least two fluid streams into the primary channel may include loading the at least two fluid streams wherein each of the at least two streams include a same antibiotic.

In accordance with some embodiments of the present invention, loading the at least two fluid streams into the primary channel may include loading the at least two fluid streams wherein each of the at least two streams include a different antibiotic.

In accordance with some embodiments of the present invention, the droplets may include an antibiotic.

In accordance with some embodiments of the present invention, the method may include lyophilizing the droplets to form a lyophilized antibiotic solute where the mass of the lyophilized antibiotic solute is related to the concentration of the antibiotic in the droplets prior to lyophilization.

There is further provided, in accordance with some embodiments of the present invention, a method for antibiotic susceptibility testing. The method for antibiotic susceptibility testing may include obtaining an antimicrobial susceptibility test (AST) kit, which may include: a microstructure formed in a substrate, the microstructure may include a primary channel with a first end and a second end, and a plurality of chambers open to the primary channel, each chamber in the plurality of chambers having a volume less than 100 nanoliters and being connected by a vent to a secondary channel in the microstructure, a width of the vent may be configured to enable a gas to escape from the chamber to the secondary channel while inhibiting the flow of a bacterial sample solution into the secondary channel, where each chamber in the plurality of chambers may include an antibiotic with a concentration of the antibiotic dependent on a position of the chamber of the plurality of chambers along the primary channel. At least one first end opening may be coupled to the first end of the primary channel and a second end opening may be coupled to the second end of the primary channel to enable the bacterial sample solution to be loaded into the device either through the at least one first end opening or the second end opening, to flow along the primary channel into the plurality of chambers, and to mix with the antibiotic in each chamber. A retaining channel may be coupled between the primary channel and the secondary channel which allows a retaining fluid in the primary channel to flow into the secondary channel while inhibiting the flow of the bacterial sample solution into the secondary channel so as to isolate droplets of the bacterial sample solution in each chamber of said plurality of chambers. The bacterial sample solution may be loaded into the primary channel and into the plurality of chambers open to the primary channel allowing the bacterial sample solution to mix with the antibiotic in the droplet in each chamber of the plurality of chambers. Upon loading the plurality of chambers with the bacterial sample solution, the retaining fluid may be loaded into the primary channel to purge the bacterial sample solution from the primary channel, and into the secondary channel so as to isolate the droplet of the bacterial sample solution with the antibiotic in each chamber of the plurality of chambers.

In accordance with some embodiments of the present invention, loading the bacterial sample solution into the primary channel may include loading the bacterial sample solution through at least two first end openings coupled to the first end of the primary channel or a second opening at the second end of the primary channel.

In accordance with some embodiments of the present invention, the antibiotic may include a lyophilized antibiotic solute.

In accordance with some embodiments of the present invention, the method for antibiotic susceptibility testing may include in an imaging system, monitoring, and acquiring data on, a growth of bacteria in the isolated droplet of bacterial sample solution in each chamber of the plurality of chambers. In a processor, the acquired data may be analyzed and information may be computed about inhibition of the growth of the bacteria based on the antibiotic and concentration of the antibiotic in the isolated droplet in each chamber of the plurality of chambers. In an output device, the information may be output.

In accordance with some embodiments of the present invention, monitoring the growth of the bacteria may include using a microscope to image bacterial cells in the isolated droplet in each chamber of the plurality of chambers.

In accordance with some embodiments of the present invention, the bacterial sample solution in the droplet may include a fluorescent indicator, and monitoring the growth of the bacteria may include analyzing fluorescence from the indicator.

In accordance with some embodiments of the present invention, the fluorescent indicator may include resazurin.

In accordance with some embodiments of the present invention, the information may include a minimal inhibitory concentration (MIC) of the antibiotic.

In accordance with some embodiments of the present invention, the information may include S/I/R determinations about the antibiotic and the bacteria.

In accordance with some embodiments of the present invention, monitoring the growth of the bacteria comprises using the imaging system to count the average number of bacteria per chamber of said plurality of chambers.

### BRIEF DESCRIPTION OF THE DRAWINGS

In order for the present invention, to be better understood and for its practical applications to be appreciated, the following Figures are provided and referenced hereafter. It should be noted that the Figures are given as examples only and in no way limit the scope of the invention. Like components are denoted by like reference numerals.

FIG. 1 schematically illustrates a microfluidic device, in accordance with some embodiments of the present invention;

FIG. 2A schematically illustrates a cross-section of a microfluidic device, in accordance with some embodiments of the present invention;

FIG. 2B schematically illustrates variants of the chambers of the microfluidic device shown in FIG. 2A, in which the chambers have narrowed entrances;

FIG. 3 schematically illustrates a steady-state, two-dimensional concentration profile map in a microfluidic device, in accordance with some embodiments of the present invention;

FIG. 4A illustrates a graph of a normalized concentration of solute in a plurality of chambers along the length of a primary channel in a microfluidic device, in accordance with some embodiments of the present invention;

FIG. 4B illustrates a graph of a normalized concentration of solute in a plurality of chambers along the length of a primary channel in a microfluidic device with varying Peclet numbers, in accordance with some embodiments of the present invention;

FIG. 5 is a flowchart illustrating a method for forming droplets with gradually varied concentrations of a solute in a microfluidic device, in accordance with some embodiments of the present invention;

FIG. 6A schematically illustrates a microfluidic device with a primary channel and chambers loaded with low concentration and high concentration fluid streams, in accordance with some embodiments of the present invention;

FIG. 6B schematically illustrates a microfluidic device loaded with a retaining fluid to purge fluid streams from a primary channel, in accordance with some embodiments of the present invention;

FIG. 6C schematically illustrates a microfluidic device with lyophilized antibiotic solute in a plurality of chambers, in accordance with some embodiments of the present invention;

FIG. 7A schematically illustrates a microfluidic device with lyophilized antibiotic solute dissolving in a bacterial sample fluid loaded into a primary channel, in accordance with some embodiments of the present invention;

FIG. 7B schematically illustrates a microfluidic device with a bacterial sample fluid being sealed by a retaining fluid loaded into a primary channel, in accordance with some embodiments of the present invention;

FIG. 7C schematically illustrates a microfluidic device for antimicrobial susceptibility testing (AST), in accordance with some embodiments of the present invention;

FIG. 8 schematically illustrates an exemplary embodiment of an antimicrobial susceptibility test (AST) kit, in accordance with some embodiments of the present invention;

FIG. 9 schematically illustrates an AST kit with at least two stationary nanoliter droplet arrays (SNDA), in accordance with some embodiments of the present invention;

FIG. 10 is schematically illustrates an AST analysis system, in accordance with some embodiments of the present invention; and

FIG. 11 is a flowchart illustrating a method for antimicrobial susceptibility testing with gradually varied concentrations of an antibiotic in a plurality of chambers in a microfluidic device, in accordance with some embodiments of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, use of the conjunction “or” as used herein is to be understood as inclusive (any or all of the stated options).

Some embodiments of the present invention described herein include methods and apparatuses for fabricating and using antimicrobial susceptibility test (AST) kits based on microfluidic devices including an array of a plurality of chambers open to a primary channel in the microfluidic device. These microfluidic devices may also be known herein as stationary nanoliter droplet arrays (SNDA) since each of the plurality of chambers may be configured to hold a volume of liquid also known as droplets on the order of nanoliters. Since the chamber volumes are small and chemically isolated, small number of bacterial cells may be detected under different antibiotic conditions for AST kits.

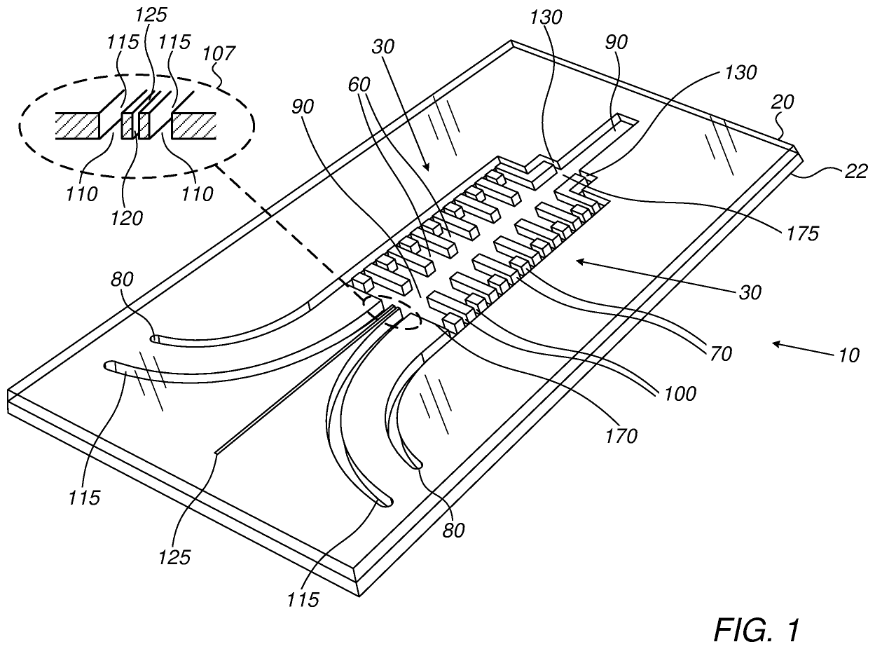

FIG. 1 schematically illustrates a microfluidic device 10, in accordance with some embodiments of the present invention. Microfluidic device 10, or stationary nanoliter droplet array (SNDA) 10, may include a second substrate 22 for sealing microfluidic device 10. Typically, second substrate 22 may permanently bonded to, removable, or may be integral to (e.g., formed of a continuous piece of material with) substrate 20. In some embodiments, substrate 20 and/or second substrate 22 may be transparent to enable viewing the interior of microfluidic device 10. Second substrate 22 may contain one or more ports or openings to enable fluids (e.g., air, sample fluid, or fluid sealant) to be introduced into or removed from the interior of microfluidic device 10 such as from the tip of a pipette, for example.

Microfluidic device 10 may include a substrate 20. Substrate 20 may be made from various materials. For example, substrate 20 may be made from a polymer, such as, for example, polydimethylsiloxane (PDMS or dimethicone), or another suitable polymer or material. Substrate 20 may include one or a plurality of microstructures 30. Microstructures 30 may include various indentations or hollowed out microstructural patterns comprising a primary channel 90, one or a plurality of secondary channels 80, chambers 60 branching off primary channel 90, and vents 100 separated by separating walls 70. Microstructures 30 may rest directly or indirectly on substrate 20. Substrate 20 may be constructed of or include the same material or a similar material to that of microstructures 30. Substrate 20 may be constructed of a distinct material from microstructures 30, for example, microstructures 30 are prefabricated and attached to a separate substrate.

Microfluidic device 10 may include inlet channels 115 (e.g., two inlet channels 115 shown in FIG. 1) and a purge channel 125 formed in substrate 20. Streams of fluids in inlet channels 115 may enter primary channel 90 at a first end 170 of primary channel 90 through an inlet channel opening 110 (e.g., two openings 110 from two respective inlet channels 115) as shown in an inset 107 of FIG. 1. Similarly, any retaining fluid in purge channel 125 may enter primary channel 90 at the first end of primary channel 90 through a purge channel opening 120 as shown in inset 107. The retaining fluid may be loaded into secondary channel 80 through one or a plurality of retaining channels 130. The width of purge channel 125 and purge channel opening 120 may be substantially smaller than width of inlet channels 115 and inlet channel opening 110 so as to provide a higher hydrostatic resistance to fluid in primary channel 90 flowing back into purge channel 125.

Microstructures 30 may be manufactured in various manufacturing processes, such as, for example a process that is associated with soft lithography. A process associated with soft lithography may include the construction of a master, e.g., in the form of a master plate or mold, using, for example, photolithography, e-beam, micro-machining, or another technique. An elastomer, such as PDMS, may be poured, spin casted, or otherwise applied to a master plate or into a mold and cured (e.g., by application of heat or ultraviolet light) or hardened. Once cured or hardened, the elastomer may be peeled away or otherwise removed from the master or mold, resulting in a set of microstructural patterns that are the inverse of those on the master. The peeled away PDMS mold may be used as a microfluidic device, or it may be used as stamp to transfer the patterns and structures of the master to another surface or platform.

In some embodiments of the present invention, microfluidic device 10 may be manufactured using a deep reactive-ion etching (DRIE) technique. DRIE is an anisotropic etching process that may be used to create deep penetration, steep-sided holes or trenches in substrates. DRIE may be cryogenic (i.e., wherein the substrate is pre-chilled prior to the chemical etching), or may use a Bosch process (pulsed or time-multiplexed etching). DRIE may enable achievement of higher resolutions that with other processes, which may enable microfluidic device 10 to operate over a wide range of pressures.

Microstructures 30 may be manufactured using other or additional processes. Microstructures 30 may be single-tiered or multi-tiered. The microstructural patterns may be configured to provide functionality for microfluidic device 10. Different microstructural patterns may be employed with one or a plurality of microfluidic devices 10, depending on the nature of the sample, reagent or other fluids (e.g., sample fluid or fluid sealant) intended to be used with microfluidic device 10. Different microstructural patterns may be used with microfluidic device 10, e.g., depending on the external environment of microfluidic device 10 or other criteria.

Microstructure 30 may include channels, pumps, valves, chambers, chambers, vents or other components in a microfluidic device.

Each chamber 60 may include an opening that connects chamber 60 to primary channel 90. A fluid may be introduced into each chamber via the primary channel (and via an opening in substrate 20 or elsewhere through which the fluid may be introduced into microstructure 30 from outside microfluidic device 10). For example, a sample fluid may be injected into an opening in substrate 20 that connects either directly or indirectly (e.g., via an intervening channel) to primary channel 90.

Prior to introduction of the fluid into the chamber, the chamber may have previously been filled by a gas (e.g., air) or by another fluid that is significantly less viscous than the sample fluid. For example, the microfluidic device, prior to filling with fluid, may have kept in a controlled atmosphere or environment from which air was excluded.

Each chamber 60 may be connected to secondary channel 80, herein referred to also as an evacuation channel, via vent 100. The evacuation channel is connected, either directly or indirectly (e.g., via an intervening channel), to an opening in the cover (or elsewhere) that opens to the ambient environment.

Vent 100 is typically located on a side of chamber 60 opposite to the opening primary channel 90, or on any other side of chamber 60 (such that vent 100 does not open to primary channel 90). Vent 100 may include an arrangement of one or a plurality of narrow slits that connect the interior of the chamber to the evacuation channel. The structure of each slit is such that the air may readily flow from the chamber into the evacuation channel through the slit. The slit is sufficiently narrow, however, to inhibit or prevent the sample fluid from exiting the chamber through the slit without application of a pressure that is appreciably greater than the pressure that is applied to introduce the sample fluid. In the absence of such greater pressure, various forces (e.g., one or more of surface tension or Laplace pressure, adhesive forces, and ambient pressure) resist motion of the sample fluid outward through the slit to the evacuation channel. Each such slit is hereinafter referred to as a vent. Thus, the vent structure includes a single vent, and may include one or a plurality of additional vents. As used herein, vent 100 is considered to inhibit flow of a fluid when, at a given pressure with which the fluid is introduced into the microstructure, flow of the fluid through the vent is prevented.

In some embodiments of the present invention, each of chambers 60 in microfluidic device 10 may include vent 100 and an evacuation channel, may be advantageous over a device with a different structure. Vent 100 may enable the air (typically at atmospheric pressure, or another gas at a pressure that is close to atmospheric pressure) to be readily evacuated from chamber 60 through vent 100 as a result of introduction of fluid into primary channel 90. In a device without such a vent and without application of high pressure, bubbles of a fluid previously filling the chamber (e.g., air) could prevent complete filling of the chamber by the sample fluid.

In some embodiments of the present invention, a solution with antibiotics, for example, or a solute of antibiotic in a fluid, or solution, may be loaded into the primary channel with at least two fluid streams through the at least two respective openings 110 at the first end of primary channel 90. The at least two fluid streams may include, for example, different concentrations of the same antibiotic solute. A combination of advection and diffusion in the at least two fluid streams may cause a concentration gradient in the antibiotic solute in the fluid mixture as the fluid mixture flows from first end 170 to a second end 175 of primary channel 90. Thus, the antibiotic solution with different concentrations of the antibiotic solute, for example, are loaded into each of the plurality of chambers 60 along the length of primary channel 90 representative of the concentration gradient of the antibiotic solute in the antibiotic solution (e.g., fluid mixture) from the first end to the second end of the primary channel.

In some embodiments of the present invention, a single concentration of a single antibiotic may be loaded into primary channel 90 through one of inlet channels 115 so as to introduce a single concentration of the antibiotic solution into each chamber in the plurality of chambers 60 (e.g., no antibiotic concentration gradients in chambers 60).

Although microfluidic device 10 shown here may be used in forming a concentration gradient along primary channel 90 for antimicrobial susceptibility testing, this is not by way of limitations of the embodiments of the present invention. The embodiments taught herein may also be used for other cytoxicity/drug screening assays such as assessing the susceptibility of cancer cells to chemotherapy. They may also be used for research applications, such as studying the effects of growth factor gradients on stem cells or monitoring T-cell activation to a number of factors, for example.

Excess antibiotic solution left in the primary channel may be purged from the primary channel using a shearing fluid such as oil or air, for example. The shearing fluid may be introduced into purge channel 125 and may enter primary channel 90 at the first end of primary channel 90 through a purge channel opening 120 as shown in inset 107. The shearing fluid may also be introduced into inlet openings 115, for example, and may enter primary channel 90. Thus, the excess antibiotic solution may be purged from primary channel 90 while retaining a plurality of droplets of the antibiotic fluid in the plurality of chambers 60. Each droplet in the plurality of droplets retained in the plurality of the chambers 60 from the first end to the second end of the primary channel may have a concentration of the antibiotic solute representative of the concentration gradient of the antibiotic solute in the antibiotic solution from the first end to the second end of the primary channel. In some cases, use of air (e.g., instead of oil) as a shearing fluid may enable reuse of the array of droplets for subsequent assays, e.g., through lyophilization (freeze drying) or by reconnecting chambers 60 with main channel 90 for an additional flow step.

In some embodiments of the present invention, the at least two fluid streams may include different antibiotics, or different antibiotic solutes in the fluid mixture and is not limited to one antibiotic, or one solute. In this manner, each of the chambers in the plurality of chambers may include different concentrations of the one or more solutes from the fluid mixture (e.g., one or more antibiotics in a mixture of different antibiotics.)

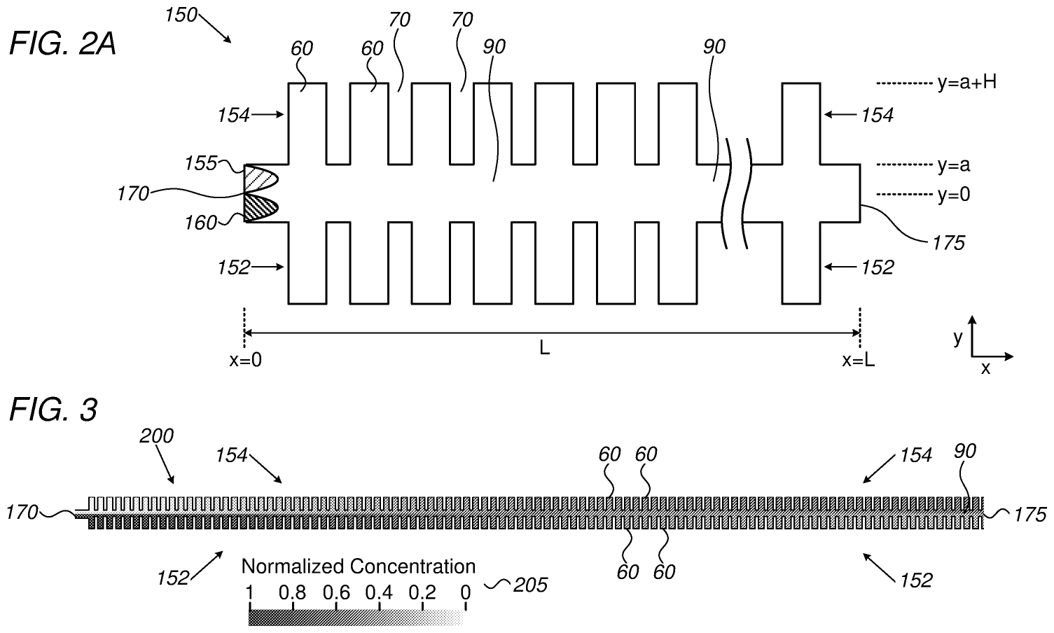

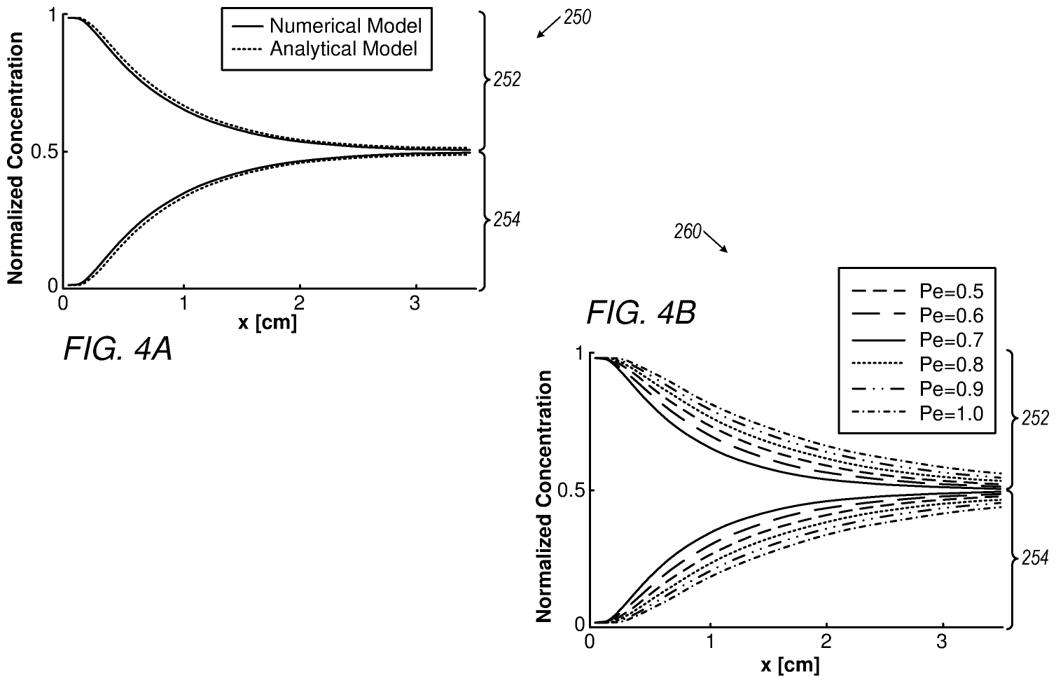

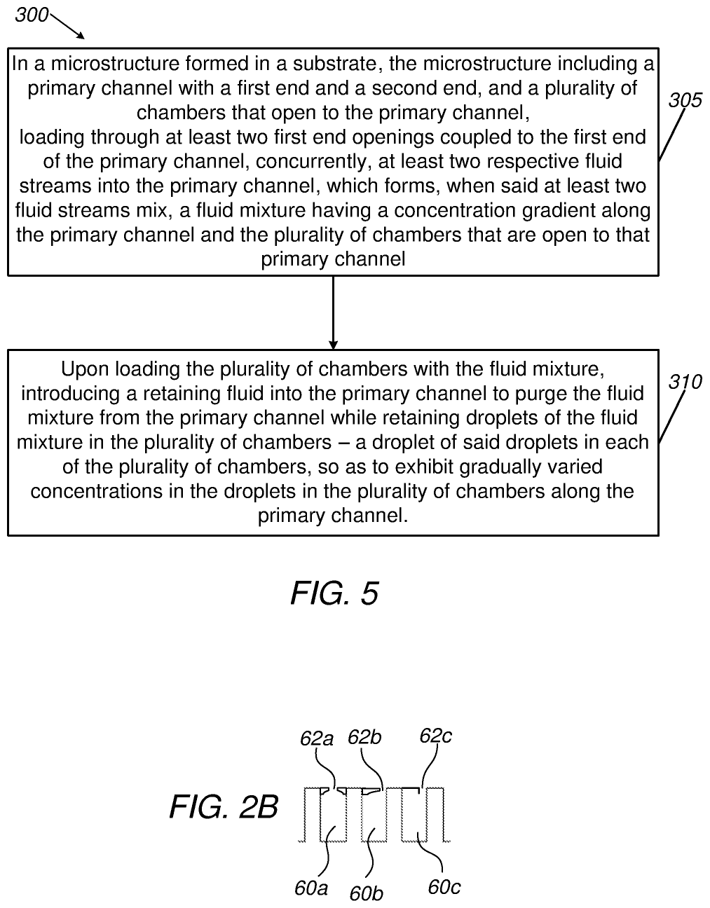

FIG. 2A schematically illustrates a cross-section 150 of microfluidic device 10, in accordance with some embodiments of the present invention. Primary channel 90 with a length L and half width a may be oriented along the x-axis as shown in FIG. 1 with the center of primary channel 90 may be placed at y=0. A first end 170 of primary channel 90 may be located at x=0 and a second end 175 of primary channel 90 may be located at x=L. FIG. 2A illustrates cross-section 150 of microfluidic device 10 in the X-Y plane.

The plurality of chambers 60 may be arranged in a first array 152 and a second array 154 where first array 152 and second array 154 of chambers 60 may be oriented along the y-axis substantially opposite to one another. Each of chambers 60 in first array 152 and second array 154 may have a height of H along the y-axis as shown in FIG. 1. Each of chambers 60 in first array 150 may be located along the y-axis from y=−a to y=−a−H. Similarly, each of chambers 60 in second array 160 may be located along the y-axis from y=a to y=a+H.

In some embodiments of the present invention, stationary nanoliter droplet array 10, which may be used in antimicrobial susceptibility test kits, may include 100-10000 chambers, for example, where each chamber in the plurality of chambers 60 may hold a fluid volume less than 100 nL, or 8 nL, for example. However, for other microfluidic applications, the fluid volume may be less than 100 nL. Each chamber in the plurality of chambers 60 branching off primary channel 90 may have dimensions of 400 μm×200 μm×100 μm (e.g., L×W×H). Note that height dimension H of chamber 60 is the dimension perpendicular to the X-Y plane shown in cross-section 150. Primary channel 90 may have dimensions of 300 μm and 100 μm corresponding to Wp=2a and Hp=H respectively.

A low concentration fluid stream 155 and a high concentration fluid stream 160 with a low concentration of solute C_{L} and a high concentration of solute C_{H}, respectively, may be introduced, or loaded, into primary channel 90 via inlet openings 115 at x=0. The two fluid streams may be merged in primary channel 90 and may mix as the two fluid streams move from first end 170 to second end 175 of primary channel 90. As the fluid mixture advects down the length of primary channel 90 (e.g., in the x-direction), the solute may diffuse along the channel width (e.g., in the y-direction).

As a result of the two fluid streams of different solute concentrations mixing in primary channel 90, a steady state gradient may develop with a concentration profile C(x,y) as long as a flow velocity U is maintained. Each chamber from the plurality of chambers 60 may sample the concentration of the solute in that section of primary channel 90 in which each chamber is in contact with. Hence, the concentration of the solute in each chamber 60 may be a function of position along the primary channel. Chambers 60 in first array 152 (e.g., closer in proximity to high concentration fluid stream 160) may sample higher to medium concentration values of solute from first end 170 to second end 175 of primary channel 90. Similarly, chambers 60 in second array 154 (e.g., closer in proximity to low concentration fluid stream 155) may sample lower to medium concentration values of solute from first end 170 to second end 175 of primary channel 90.

An analytical model was developed by the inventors to describe the concentration profile C(x,y) in primary channel 90 in steady state. For a diffusion coefficient of the solute, D, the model may assume a fully developed flow field at steady state to account for mass transport in primary channel 90 using a two-dimensional advection-diffusion equation with advection only in the \hat{x}-direction as described in equation (1):

$$\frac{\partial C}{\partial t} = - {u}_{x} \frac{\partial C}{\partial x} + D ( \frac{{\partial }^{2} C}{\partial {x}^{2}} + \frac{{\partial }^{2} C}{\partial {y}^{2}} ) \qquad ( 1 )$$

For gradient production, advection in the \hat{x}-direction may be assumed to be on the same time scale as diffusion only in the ŷ-direction, and axial diffusion in the \hat{x}-direction may be assumed to be negligible such that equation 1 may be simplified to:

$$\frac{\partial C}{\partial t} = - U \frac{\partial C}{\partial x} + D \frac{{\partial }^{2} C}{\partial {y}^{2}} \qquad ( 2 )$$

Adopting an Euler specification of the flow field and factoring the appropriate boundary conditions, equation (2) may be solved to yield a gradient concentration profile of solute in primary channel 90 as follows in Equation (3), where the erf operator is the Gaussian error function:

$$C ( x , y ) = \frac{{C}_{H}}{2} [ erf ( \frac{y - a}{2 \sqrt{D \frac{x}{U}}} ) - erf ( \frac{y - 3 a}{2 \sqrt{D \frac{x}{U}}} ) + erf ( \frac{y - 5 a}{2 \sqrt{D \frac{x}{U}}} ) - erf ( \frac{y - 7 a}{2 \sqrt{D \frac{x}{U}}} ) - erf ( \frac{y + a}{2 \sqrt{D \frac{x}{U}}} ) + erf ( \frac{y + 3 a}{2 \sqrt{D \frac{x}{U}}} ) ] \qquad ( 3 )$$

In some cases, it may be advantageous to provide a narrow entrance from primary channel 90 to each chamber of microfluidic device 10. For example, narrowing the entrance may prevent or inhibit advection during loading from actively mixing reagents from one chamber to another.

FIG. 2B schematically illustrates variants of the chambers of the microfluidic device shown in FIG. 2A, in which the chambers have narrowed entrances;

In the examples shown, chambers 60a-60c are provided with narrow openings 62a to 62c, respectively, to channel 90. For example, narrow opening 62a is symmetric, while narrow openings 62b and 62c are asymmetric. Narrow openings 62a and 62b are provided with wedge-like narrowing structure, while narrow opening 62c is provided with a flat narrowing structure. Combinations of features of the above, or other configurations of narrowing structure, may be used.

FIG. 3 schematically illustrates a steady-state, two-dimensional concentration profile map 200 in microfluidic device 10, in accordance with some embodiments of the present invention. Profile map 200 illustrates the relative concentrations of the solute samples by each chamber in the plurality of chambers 60 in microfluidic device 10 based on the concentration gradient from the advection-diffusion of the solute in the at least two fluid streams with different concentrations (e.g., low concentration fluid stream 155 and high concentration fluid stream 160). A mapping key 205 illustrates the normalized concentration of solute in the fluid mixture in primary channel 90 generated from simulations based on the analytical model of Equation (3). First array 152 of chambers 60 may include the higher concentrations of solute sampled at first end 170 to medium concentrations of solute sampled at second end 175 of primary channel 90. Second array 154 of chambers 60 may include the lower concentrations of solute sampled at first end 170 to medium concentrations of solute sampled at second end 175 of primary channel 90 as shown in FIG. 3 and described previously.

FIG. 4A illustrates a graph 250 of a normalized concentration of solute in plurality of chambers 60 along the length of primary channel 90 of microfluidic device 10, in accordance with some embodiments of the present invention. Graphs 252 are the normalized concentrations in first array 152 of chambers 60 along the length of the primary channel from x=0 to x=L. Graphs 254 are the normalized concentrations in second array 154 of chambers 60 along the length of the primary channel from x=0 to x=L. FIG. 4A compares the analytical model of equation (3) with a numerical model based on a computational model of the 2D advection-diffusion equation (1) in the time domain. The numerical model in shown in graphs 252 and 254 in FIG. 4A are the steady state solutions of the computational time domain model showing agreement with the analytical model of Equation (3).

FIG. 4B illustrates a graph 260 of a normalized concentration of solute in plurality of chambers 60 along the length of primary channel 90 of microfluidic device 10 with varying Peclet numbers, in accordance with some embodiments of the present invention. The Peclet number Peₐ, a unitless parameter, which may be used to describe the ratio of the advective transport rate to the diffusive transport rate of the solute in the fluid mixture flowing in primary channel 90 given an average flow velocity of U. The Peclet number is given by Peₐ=UD/La². For Pe=0.5, nearly every normalized concentration of the solute in the concentration gradient profile may be sampled in each chamber in first array 152 and second array 154 of chambers 60 from x=0 to x=L along the length of primary channel 90. Stated differently, each chamber in the plurality of chambers 60 will sample concentration of solutes between the chosen limits of C_{H} and C_{L} In the embodiment shown in FIGS. 4A and 4B, C_{L}=0 in normalizing from 0 to 1.

The concentration of the solute sampled by each chamber may be accurately determined by the computational analyses described above. In the previous analyses, one solute and two fluid streams were used in the previous analysis for conceptual clarity. However, this is not by way of limitation of the embodiments of the present invention in that any number of solutes in at least two fluid streams may be introduced into primary channel 90 to form the fluid mixture with a concentration gradient in accordance with the embodiments described in FIGS. 1-4. These methods may be used, for example, in AST kits where the solute sampled in each chamber 60 may include an antibiotic where the concentration of the antibiotic may be accurately determined in each chamber in the plurality of chambers 60 in microfluidic device 10. Moreover, a different antibiotic may be used in each of the at least two fluid streams, for example, such that the concentration of the solute in each chamber may be a combination of one or more antibiotics.

FIG. 5 is a flowchart illustrating a method 300 for forming droplets with gradually varied concentrations of a solute in microfluidic device 10, in accordance with some embodiments of the present invention. Method 300 includes in microstructure 30 formed in substrate 20, the microstructure including primary channel 90 with first end 170 and second end 175, and a plurality of chambers 60 that open to primary channel 90, loading 305 through at least two first end openings 110 coupled to first end 170 of primary channel 90, concurrently, at least two respective fluid streams 155, 160 into primary channel 90, which forms, when said at least two fluid streams mix, a fluid mixture having a concentration gradient along primary channel 90 and the plurality of chambers 60 that are open to that primary channel 90.

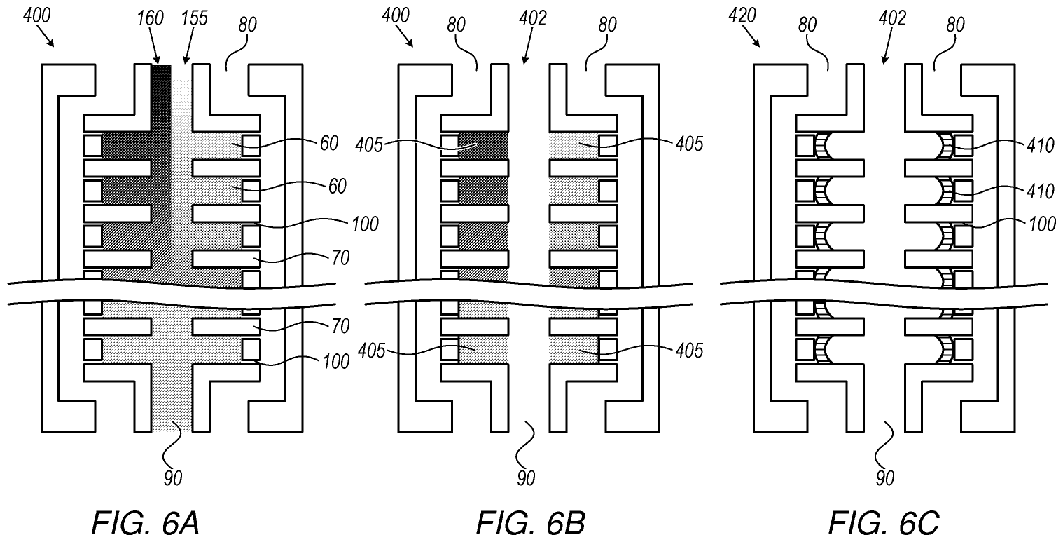

Method 300 includes upon loading 305 the plurality of chambers 60 with the fluid mixture, introducing 310 a retaining fluid 402 (see FIG. 6B) into primary channel 90 to purge the fluid mixture from primary channel 90 while retaining a droplets 405 (see FIG. 6B) of the fluid mixture in the plurality of chambers 60—a droplet of said droplets 405 in each of the plurality of chambers, so as to exhibit gradually varied concentrations in the droplets 405 in the plurality of chambers 60 along the primary channel 90.

The embodiments for forming droplets with gradually varied concentrations in a microfluidic device as shown in FIGS. 1-3 are just by way of example and not by way of limitation of the embodiments of the present invention. For example in some embodiments, the at least two fluid streams 155, 160 in at least two respective external tubes may be joined and mixed in a single external tube, which may be introduced into primary channel 90 via one inlet channel opening (e.g., through one of the inlet channel openings 110.

FIG. 6A schematically illustrates a microfluidic device 400 with primary channel 90 and chambers 60 loaded with low concentration 155 and high concentration 160 fluid streams, in accordance with some embodiments of the present invention.

FIG. 6B schematically illustrates microfluidic device 400 loaded with a retaining fluid 402 to purge the fluid streams from primary channel 90, in accordance with some embodiments of the present invention. Retaining fluid 402, such as air, for example, may be injected through purge channel 125 and/or channels 115 at first end 170 of primary channel 90 or injected through second end 175 of primary channel 90 while retaining and isolating a plurality of droplets 405 in the respective plurality of channels 60. The plurality of droplets 405 may include gradual varied concentrations of the solute indicative of the concentration gradient formed by the advection and diffusion of the solutes in the at least two fluid streams.

In using microfluidic device 10 for AST kits, each of droplets 405 may include one or a plurality of antibiotic solutes, for example. If the antibiotic droplets remain in liquid form, the effectiveness of the antibiotics may degrade over time. Thus, once the plurality of chambers is loaded with the antibiotic droplets, the droplets are lyophilized, or freeze-dried, so as to produce a lyophilized antibiotic solute such that AST kits may be stored for longer period of time.

FIG. 6C schematically illustrates microfluidic device 420 with lyophilized antibiotic solute 410 in the plurality of chambers 60, in accordance with some embodiments of the present invention. To lyophilize antibiotics droplets 405 within each chamber 60 of SNDA 10, the arrays of chambers may be frozen, for example, at −80° C. for 40 minutes, and may then be subsequently placed into vacuum chambers for overnight lyophilization in a lyophilizer machine.

After lyophilization, lyophilized antibiotic solute 410 as shown in FIG. 6C may remain in each chamber of the plurality of chambers 60 with a mass of the lyophilized antibiotic solute proportional to the concentration of the solute in the droplet prior to lyophilization, where the mass of the antibiotics in each of the chambers are controlled and accurately known in accordance with the analytic model of equation (3), for example.

In some embodiments of the present invention, after fabricating AST kits with SNDA 10 including a gradually varied mass of lyophilized antibiotics in each of the plurality of chambers 60, bacteria in a sample fluid may be injected into primary channel 90 and into each of chambers 60. The lyophilized antibiotic solute may dissolve in the bacterial sample fluid. Vents 100 allow air in the sample fluid to pass into secondary channel 80 while inhibiting the flow of the sample fluid into secondary channel 80.

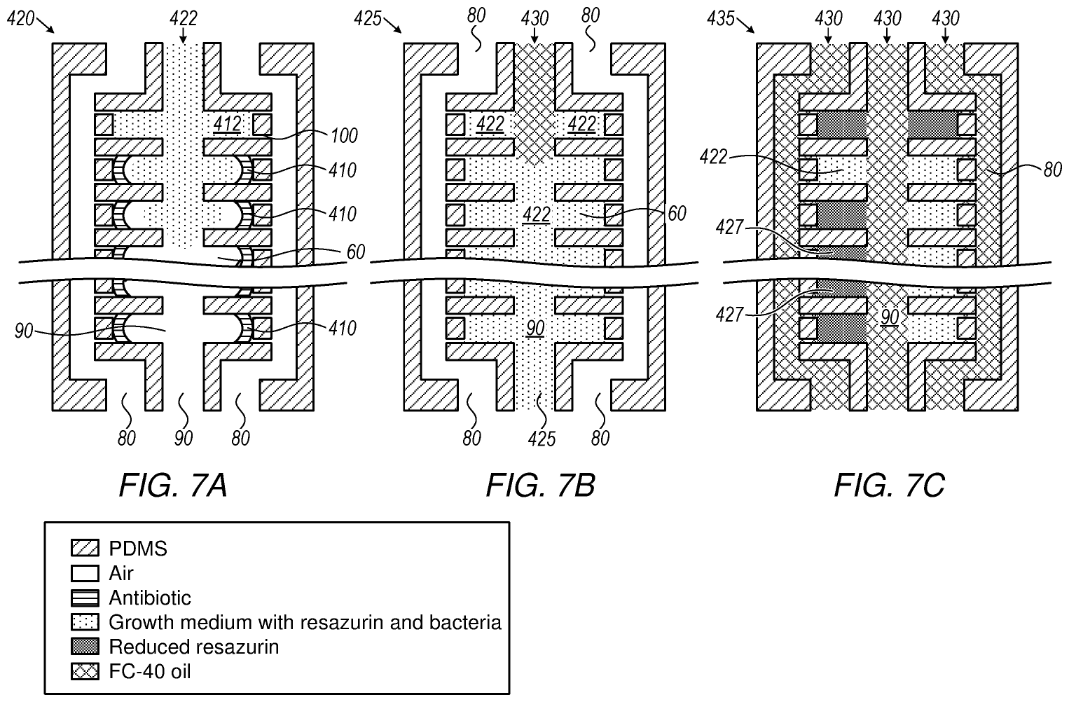

FIG. 7A schematically illustrates microfluidic device 420 with lyophilized antibiotic solute 410 dissolving in a bacterial sample fluid 422 loaded into primary channel 90, in accordance with some embodiments of the present invention.

FIG. 7B schematically illustrates a microfluidic device 425 with bacterial sample fluid 422 being sealed by a retaining fluid 430 loaded into primary channel 90, in accordance with some embodiments of the present invention. Upon filling primary channel 90 and each chamber 60 with sample bacterial fluid 422, retaining fluid 430 such as FC-40 oil may be loaded into primary channel 90 purging bacterial sample fluid 422 from primary channel 90. With reference to FIG. 1, both bacterial sample fluid 422 and FC-40 oil 430, for example, may be introduced through channel 125, 90, and/or 115.

At the second end of primary channel 90, only FR-40 oil 430, or a substance of significantly lower surface tension may pass though one or a plurality of retaining channels 130 into secondary channels 80. The gap size of retaining channels 130 is configured to inhibit the flow of bacterial sample fluid 422 into secondary channels 80 based on the surface tension of sample fluid 422 which creates Laplace pressure on the sample interface at the one or a plurality of retaining channels 130, but allows FR-40 oil 430 having significantly lower surface tension than sample fluid 422 to pass through the one or a plurality of retaining channels 130. In this manner, the bacterial sample fluid droplets may be completely sealed in each chamber 60 by an immiscible barrier due to the retaining fluid in primary channel 90 and secondary channel 80. However, secondary channels 80 may also be filled by any suitable from an external inlet channel formed in microstructure 30.

FIG. 7C schematically illustrates a microfluidic device 435 for antimicrobial susceptibility testing, in accordance with some embodiments of the present invention. In antimicrobial susceptibility testing, a known mass of lyophilized antibiotic solute may be dissolved in bacterial sample fluid droplets in each of the sealed chambers 60. Without the dissolved antibiotic solute, the number of bacteria in the droplet sealed in a given chamber with a volume of about 8 nL, for example, may grow and proliferate. Typically, the bacterial sample fluid may be characterized by the number of bacterial colony forming units (CFU) per unit volume.

An antibiotic may be classified as either bacteriostatic or bactericidal. In bacteriostatic antibiotics, the number of bacteria may remain static or does not increase. In bactericidal antibiotics, the bacteria are killed within the sealed chamber. In either case, the growth of the bacteria may be monitored optically by observing the number of bacteria under a high power microscope or by using other optical methods, such as fluorescence in conjunction with secondary reporters, to identify if the number of bacteria increase and/or to assess the state of the bacterial culture within each droplet sealed in the plurality of chambers 60.

In rapid antimicrobial susceptibility testing, the number of bacteria in the chambers may be monitored and sampled at predetermined time intervals. Statistical analyses may be applied to the bacterial colony data to determine if enough time has elapsed since sealing the droplet to assess whether a particular mass and/or concentration of the antibiotic has been successful in inhibiting bacterial growth, and what is the breakpoint or threshold mass and/or of the antibiotic to determine the therapeutic success or failure in inhibiting bacterial growth. This approach using in the AST kits shown herein provides much less time in assessing therapeutic success or failure in inhibiting bacterial growth relative to standard AST approaches.

In some embodiments of the present invention, the breakpoint, or threshold mass, or concentration may be known as the minimum inhibitory concentration (MIC) of a given antibiotic acting on a given bacterial genotype. A standardized, threshold-based assessment scheme may be used in which the degree of antibiotic, or drug, effectiveness may be characterized as “susceptible”, “intermediate”, and “resistant” (e.g., S/I/R determination) depending on the MIC value. Note that concentration of the antibiotic and the mass of the lyophilized antibiotic solute are metrics that may be used interchangeably, since the volume of chambers 60 remains constant, so the concentration and mass of the antibiotic are directly and linearly correlated with a known constant.

In some embodiments of the present invention, the AST kit (e.g., microfluidic device 435) may be optically transparent. Each chamber 60 may be labeled with an identification number with the type of antibiotic, concentration of the antibiotic and/or mass of the lyophilized antibiotic known a priori in each chamber, for example. Microfluidic device 435 may be placed under a microscope and/or an imaging system with sufficient magnification and may be configured to image each chamber in the plurality of chambers 60 at the predefined time intervals. Image processing techniques may be used to determine the number of CFUs/volume, or to assess the state of the bacterial culture using some correlative parameter/reporter in each of the chambers. Systems used to image bacteria in each of the plurality of chambers typically need magnifications of 600 while the image systems described herein may use magnification of 10.

In some embodiments of the present invention, a processing unit, such as a computer, may be configured to analyze to the number of CFUs/volume or some correlative parameter with the concentration and/or mass of the antibiotic in each of chambers 60 in each of the predefined time intervals. The MIC and S/I/R determinations about the antibiotic may be determined from this data.

In some embodiments of the present invention, the bacterial cells are not imaged. However, a molecular or chemical indicator may be introduced in the bacterial sample fluid and subsequently into the sealed droplet, where a property of the indicator may change based on some input by the bacteria. For example, the presence of bacteria may cause the indicator to fluoresce upon reduction by the metabolic enzymes of the bacteria and/or may cause the indicator to change color according to the pH of the medium, for example. The greater the intensity of fluorescence may be proportional to the number of CFUs/volume.

In some embodiments of the present invention, the bacterial sample fluid may be mixed with resazurin for allowing a user or an imaging system to optically detect whether the dosage, or mass, of the lyophilized antibiotic mixing with the sample fluid in a given chamber kills the bacteria. Resazurin is both a colorimetric and fluorescent dye that is minimally toxic and commonly used for cell viability assays.

The irreversible reaction of resazurin to resorufin in the sample fluid may result in a color change in the sample fluid from blue to red, in addition to the reduced molecule exhibiting red fluorescence unlike its unreduced counterpart. Resorufin fluoresces when exposed to green light. In a growth culture, this reaction occurs at a rate proportional to that of the aerobic respiration of cells in the medium. Due to the high sensitivity of fluorescent detection systems, resazurin may be used to monitor the viability of individual bacteria without the need for imaging individual cells. This may bypass the need for high-resolution optics and may permit high throughput scanning and parallelization.

In some embodiments of the present invention, bacterial sample fluid 422 including a molecular or chemical indicator, such as resazurin, for example, may be loaded into the microfluidic device, mixing with the lyophilized antibiotic 410 and bacterial sample droplets sealed in each chamber 60 by retaining fluid 430 (e.g., FR-40 oil). Each chamber 60 in the microfluidic device 435 may be monitored at predefined time intervals for changes in the state of the resazurin as the antibiotic affects the bacterial sample fluid. Droplets in which there are no or low levels of bacterial growth, the state of the resazurin may remain unchanged e.g., similar in color and fluorescence levels as in bacterial sample fluid 422 where the antibiotic and concentration of antibiotic may be therapeutically successful in inhibiting bacterial growth. However, some droplets in the chambers shown in FIG. 7C may have a proliferation of bacterial growth indicating that the antibiotic and/or the antibiotic concentration may be therapeutically ineffective with reduced resazurin (e.g., reduced resazurin droplets 427) exhibiting a higher fluorescence intensity.

Microfluidic device 435 may be placed in an imaging system configured to illuminate the sample with green light, for example, and to image the fluorescence from the reduced resazurin in each chamber in the plurality of chambers 60 at predefined time intervals. Image processing techniques may be used to determine the number of CFUs/volume in each of the chambers.

In some embodiments of the present invention, a processing unit, such as a computer, may be configured to analyze to the number of CFUs/volume with the concentration and/or mass of the antibiotic in each of chambers 60 in each of the predefined time intervals. The MIC and S/I/R determinations about the antibiotic may be determined from this data.

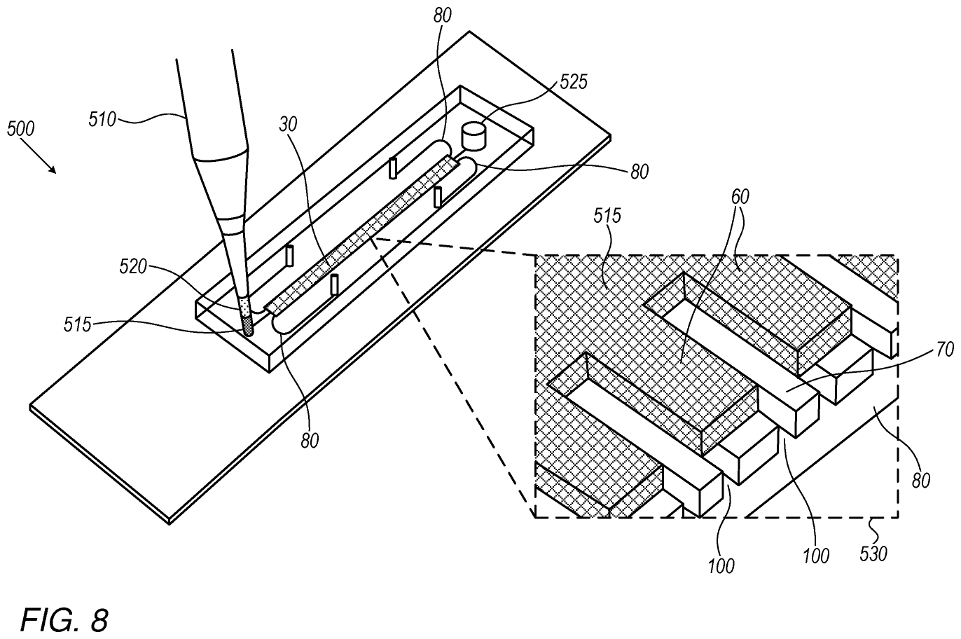

FIG. 8 schematically illustrates an exemplary embodiment of an antimicrobial susceptibility test (AST) kit 500, in accordance with some embodiments of the present invention. AST kit 500 may include SNDA 10 with microstructure 30 as shown in FIG. 1 as a base platform for simple well loading and stationary droplet formation. Each array (e.g., first array 152 and second array 154) may include 100 chambers, each holding a volume of 8 nL, and each chamber open to primary channel 90. The dimensions of chamber 60 may be 200 μm×400 μm×100 μm (W×L×H), for example, and primary channel 90 is 300 μm wide while vents 100 are 2-5 μm wide. The volume of chamber 60 may be set so that the standard AST cell concentration (5×10⁵ CFU/mL) would produce an average of 4 CFUs per chamber.

A bacterial sample fluid 515 and a FC-40 oil 520 (e.g., retaining fluid) may be loaded with a single-step injection of a two-plug solution using a conventional laboratory micropipette 510. Bacterial sample fluid 515 may be a ˜1.6 μL bacterial suspension of 5×10⁵ CFU/mL including with 10% Resazurin, for example. FC-40 oil 520 with a volume of about ˜3 μL may be used. The two-plug solution is achieved simply by aspirating the respective fluids sequentially into micropipette 510.

The two-plug solution shown in FIG. 8 may be sequentially loaded into primary channel 90 via purge channel 125 or via an opening 525 at the second end of primary channel 90. Upon discharging the two-plug solution in micropipette 510 through purge channel 125, bacterial sample fluid 515 (e.g., the first plug) may be loaded into primary channel 90 and into each chamber in the plurality of chambers 60. Low pressure loading may be enabled by vents 100 in each of chambers 60, allowing the air in the chambers to escape through vents 100 into secondary channels 80, being gradually replaced by bacterial sample fluid 515, as shown in an enlargement 530 of FIG. 8. Thus, manual low pressure loading using micropipette 510, for example, may be possible in this manner.

In some embodiments of the present invention, each chamber in the plurality of chambers 60 may include arrays with gradually varied masses of lyophilized antibiotic. Lyophilized antibiotic solute 410 as shown in FIG. 6C, for example, do not inhibit the movement of air through vents 100.

In some embodiments of the present invention, each chamber in the plurality of chambers 60 may include arrays with gradually varied concentrations of antibiotic fluids (e.g., not freeze-dried).

After the first plug with bacterial sample fluid 515 flows into primary channel 90 and into chambers 60, the second plug of retaining fluid including FC-40 oil 520 may flow into primary channel 90 and may separate chambers 60 with an immiscible barrier, effectively discretizing the bacterial sample fluid 515 into isolated droplets with a known concentration of antibiotics dissolved therein. Furthermore, as FC-40 oil 520 flows down primary channel, FC-40 oil 520 passes through retaining channels 130 into secondary channels 80 at the second end of primary channel (see FIG. 1) and fills secondary channels, isolating the droplets in chambers 60 from both sides as in FC-40 oil 430 shown in FIG. 7C. FC-40 oil 520 is a fluorinated oil that may deliver dissolved oxygen to each of the bacterial droplets in the isolated chambers while preventing evaporation of the fluid in chambers 60.

Once AST kit 500 is loaded, the growth or inhibition of bacterial colonies in each of the isolated droplets may then be monitored at predefined intervals for assessing bacterial number and proliferation in this assay.

In some embodiments of the present invention, bacterial number and proliferation data may obtained by analyzing the fluorescence within each chamber 60 for different antibiotic conditions, for example.

In some embodiments of the present invention, positive and negative control data may be used as references for assessing bacterial number and proliferation in this assay. Positive control data may include bacterial sample fluid droplets without antibiotics for assessing the highest level of bacteria metabolism or proliferation possible in AST kit 500. Conversely, negative control data may include bacterial sample fluid droplets with very high concentrations of antibiotics so as to assess the lowest possible level of bacteria metabolism or proliferation possible in AST kit 500. The bacterial number and proliferation data routine antimicrobial susceptibility testing may then be compared to the positive and negative control data references.

In some embodiments of the present invention, an imaging system may be used to image the bacterial cells in each chamber 60 in AST kit 500 and image processing techniques may be executed using a processing unit to count the number of bacterial cells per chamber 60. Positive and negative control data references may be needed to analyze the data.

For the bacteriostatic and/or bactericidal antibiotics, the baseline (e.g., negative control data references) may be higher for bacteriostatic antibiotics, but the methods for data extraction and analysis may be the same for both bacteriostatic and bactericidal antibiotics.

In some embodiments of the present invention, for the case of bacterial number and proliferation data using the relative fluorescence intensity of each of chamber in SNDA 10, relative bacterial number values and/or slopes of the bacterial number values at every predefined time interval may be acquired and analyzed. This data may be smoothed by applying any suitable fitting function.

In some embodiments of the present invention, different antibiotic concentrations in each chamber of the plurality of chambers 60 may be tested. The MIC (minimal inhibitory concentration) may include the lowest antibiotic concentration that may be shown to inhibit the proliferation and metabolism of the bacteria by a predefined threshold of 90% or more, for example, as compared to the positive control data reference normalized to the negative control data reference. From here, these MIC values may be interpreted into S/I/R determinations, which may be an accurate and quantitative method to perform antimicrobial susceptibility testing.

In some embodiments of the present invention, a single “critical” or breakpoint antibiotic concentration may be tested. If the proliferation and metabolism of the bacterial colonies may be inhibited a predefined threshold of 90% or more, for example, as compared to the positive control data reference normalized to the negative control data reference, then the bacteria may be considered susceptible. If not, then the bacteria may be considered resistant. This approach may not be an optimal approach to perform antimicrobial susceptibility testing limiting results to two S/I/R categories (susceptible/resistant). Health care professionals such as doctors may not be able to assess the level of resistance when comparing different antibiotics. For example, if a particular strain of E. coli bacteria may be resistant to both Ampicillin (AMP) and Ciprofloxacin (CIP). However, the MIC for AMP is 128 mg/L and the MIC for CIP is 16 mg/L. The doctor may not be able to access that E. coli may be highly resistant to AMP, but moderately resistant to CIP.

AST kit 500 as shown in FIG. 8 may provide rapid, same day AST results using two orders of magnitude less reagents by monitoring the bacterial growth at predefined intervals in each of the isolated and sealed nanoliter chambers in the plurality of chambers 60 for reducing cost and reliability in antimicrobial susceptibility testing, pertaining to the reliability of antibiotic sources. Loading the sample can be easily achieved by hand, with a single step injection using a conventional laboratory pipette (e.g., micropipette 510), useful for low-cost settings by reducing its dependency on large and expensive laboratory equipment.

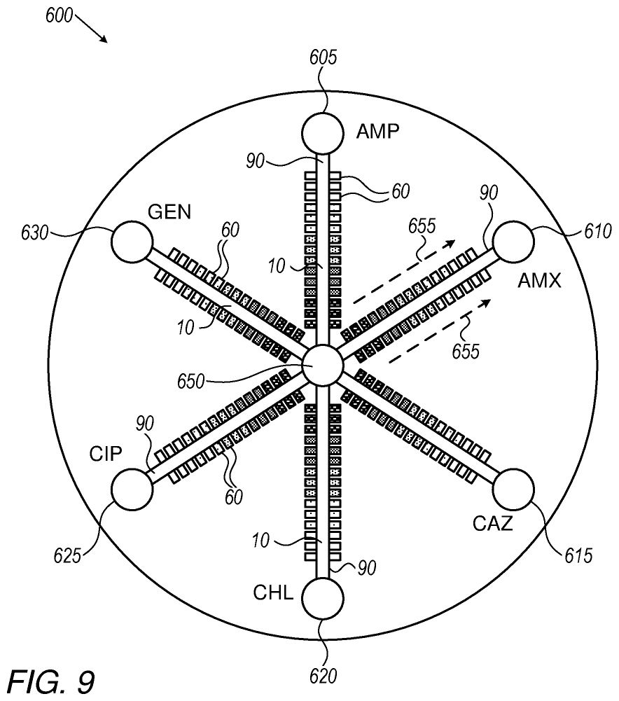

FIG. 9 schematically illustrates an AST kit 600 with at least two stationary nanoliter droplet arrays (SNDA) 10, in accordance with some embodiments of the present invention. AST kit 600 with six SNDAs 10, for example, may multiplex a plurality of different antibiotics (e.g., in liquid or lyophilized form) with pre-loaded with graded varied concentrations of the plurality of different antibiotics formed by method 300, for example, so as to simultaneously test a bacterial sensitivity in a bacterial sample fluid to the plurality of antibiotics.

For example, AST kit 600 with six SNDAs 10 may be pre-loaded with varied gradient concentrations in each chamber 60 with six antibiotics, for example: ampicillin (AMP) 605, amoxicillin (AMX) 610, ceftazidime (CAZ) 615, chloramphenicol (CHL) 620, ciprofloxacin (CIP) 625, and gentamicin (GEN) 630. Micropipette 510 with two-plug solution of bacterial sample fluid 515 and retaining fluid 520 (e.g., FC-40, for example) may be used to load bacterial sample fluid 515 via a common opening 650 into each SNDA 10. Bacterial sample fluid 515 may flow in a direction in arrows 655 in primary channel 90 and into each chamber 60 of the at least two SNDA 10 as shown in FIG. 9.

With such SNDA parallelization schemes such as shown in FIG. 9 using the at least two SNDAs 10, bacterial number and proliferation data may be acquired in each chamber 60 in the plurality of chambers 60 in each SNDA 10. An algorithm (e.g., running on a processing unit) for automating an analysis of the bacterial number and proliferation data and making S/I/R determinations for analyzing the bacterial growth in each chamber 60. The SNDA-AST system described above may reduce bacterial sample solution preparation time and perform AST directly on bacteria harvested from the bacterial sample solution. Bypassing a solid phase incubation step (e.g., plating step) in bacterial sample solution preparation may save up to 2 days of clinical diagnostic time.

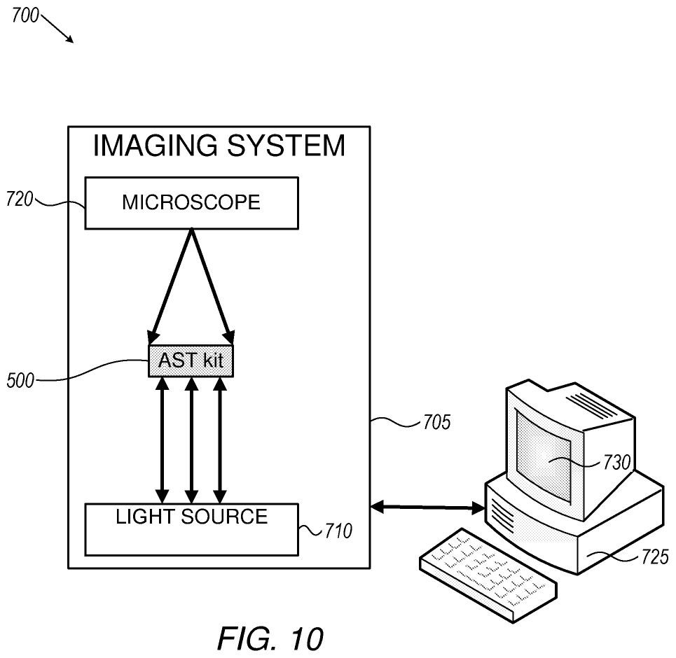

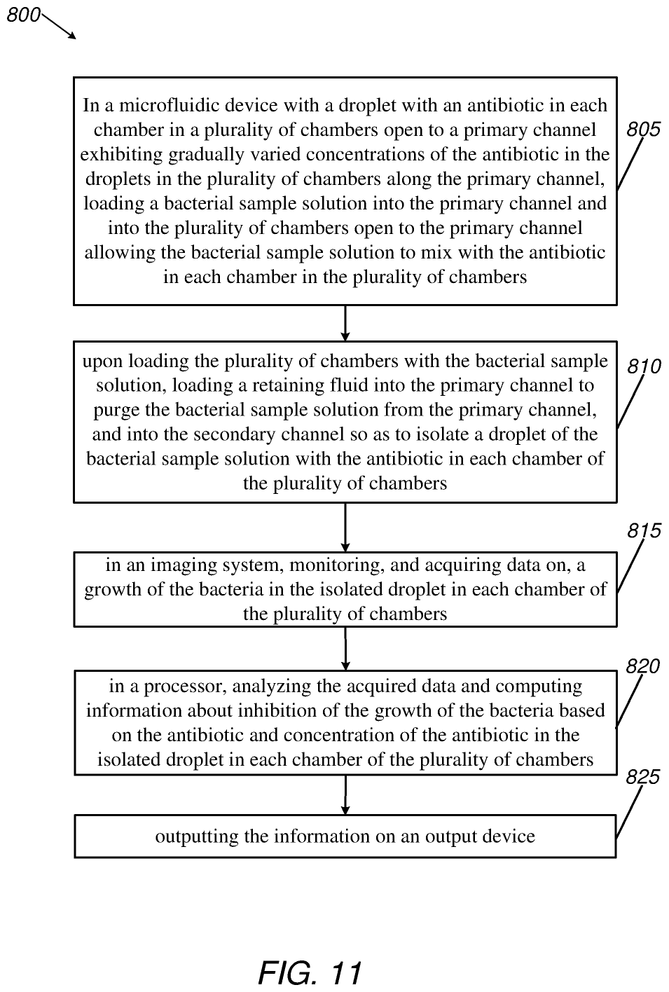

FIG. 10 is schematically illustrates an AST analysis system 700, in accordance with some embodiments of the present invention. System 700 may include an imaging system 705 including an optical microscope 720 on which AST kit 500 may be placed. Imaging system 705 may be configured to receive imaging data from microscope 720. In some embodiments, imaging system may illuminate the plurality of bacterial droplets with the antibiotic isolated in the plurality of chambers 60 in AST kit 500 with an optical fluorescence light source in a fluorescence unit 710. Fluorescence unit 710 may be configured to measure the intensity of the fluorescence from an indicator in the droplets (e.g., resazurin) indicative of the growth of bacteria in each of the imaged droplets.

Imaging system 705 may be configured to monitor, and to acquire data on, a growth of the bacteria in the isolated droplets in each chamber of the plurality of chambers 60 in AST kit 500.

In some embodiments of the present invention, system 700 may include a processing unit 725 (e.g., a processor) configured to analyze the acquired data and to compute information about inhibition of the growth of the bacteria based on the antibiotic and concentration of the antibiotic in the isolated droplet in each chamber of the plurality of chambers.

In some embodiments of the present invention, system 700 may include an output device 730, such as a monitor 730, for outputting the computed information.

FIG. 11 is a flowchart illustrating a method 800 for antimicrobial susceptibility testing with gradually varied concentrations of an antibiotic in a plurality of chambers 60 in microfluidic device 10, in accordance with some embodiments of the present invention.

Method 800 may be performed using microstructure 30 formed in substrate 20, microstructure 30 may include primary channel 90 with first end 170 and second end 175, and the plurality of chambers 60 open to primary channel 90, where each chamber in the plurality of chambers includes an antibiotic exhibiting gradually varied concentrations of the antibiotic in the droplets in the plurality of chambers 60 along the primary channel 90.

Method 800 may include loading 805 a bacterial sample solution into primary channel 90 and into the plurality of chambers 60 open to primary channel 90 allowing the bacterial sample solution to mix with the antibiotic in each chamber in the plurality of chambers 60.

Upon loading 805 the plurality of chambers with the bacterial sample solution, method 800 may include loading 810 a retaining fluid into the primary channel to purge the bacterial sample solution from the primary channel, and into the secondary channel so as to isolate a droplet of the bacterial sample solution with the antibiotic in each chamber of the plurality of chambers.

In imaging system 705, method 800 may include monitoring 815, and acquiring data on, a growth of the bacteria in the isolated droplet in each chamber of the plurality of chambers. For example, a bacterial sample, which may be isolated directly from a patient sample, may be loaded into microstructure 30, for detection of bacteria and estimation of the bacterial concentration by counting the average number of bacteria per chamber using imaging system 705.

In processor 725, method 800 may include analyzing 820 the acquired data and computing information about inhibition of the growth of the bacteria based on the antibiotic and concentration of the antibiotic in the isolated droplet in each chamber of the plurality of chambers.

Method 800 may include outputting 825 the computed information on output device 730 (e.g., a monitor).

In some embodiments of the present invention, the computed information may include the MIC of the antibiotic for a given bacterial genotype in the bacterial sample solution and S/I/R determinations about the antibiotic and the given bacterial type and/or bacterial identity.

In some embodiments of the present invention, method 800 may include loading 805 the bacterial sample solution through one or more of the at least two first end openings coupled to the first end of the primary channel or the second opening at the second end of the primary channel.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

Different embodiments are disclosed herein. Features of certain embodiments may be combined with features of other embodiments; thus certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A microfluidic device, comprising:
a microstructure formed in a substrate, the microstructure comprising a primary channel with a first end and a second end, and a plurality of chambers that open to the primary channel;
at least two openings coupled to the first end of the primary channel, to load at least two fluid streams into the device through the first end of the primary channel to flow along the primary channel from the first end to the second end into the plurality of chambers, each chamber of the plurality of chambers having a volume less than 100 nanoliters and being connected by a vent to a secondary channel in the microstructure, a width of the vent configured to enable a gas to escape from the chamber to the secondary channel while inhibiting the flow of said at least first and second fluid streams into the secondary channel; and
one or a plurality of retaining channels coupled between the primary channel and the secondary channel to allow a retaining fluid in the primary channel to flow into the secondary channel while inhibiting the flow of fluid of said at least two fluid streams into the secondary channel.

2. The microfluidic device according to claim 1, further comprising a second end opening coupled to the second end of the primary channel, and wherein the retaining fluid may be loaded into the primary channel.

3. The microfluidic device according to claim 1, wherein the plurality of chambers that open to the primary channel are arranged in a first array of chambers from the plurality of chambers positioned along a first side of the primary channel and a second array of chambers from the plurality of chambers positioned along a second side of the primary channel substantially opposite to the first array.

4. The microfluidic device according to claim 1, wherein the vent comprises one or a plurality of slits.

5. The microfluidic device according to claim 1 wherein an opening between a chamber of said plurality of chambers and the primary channel includes narrowing structure.

6. An antimicrobial susceptibility test (AST) kit, comprising:
a microstructure formed in a substrate, the microstructure comprising a primary channel with a first end and a second end, and a plurality of chambers open to the primary channel, each chamber in the plurality of chambers having a volume less than 100 nanoliters and being connected by a vent to a secondary channel in the microstructure, a width of the vent configured to enable a gas to escape from the chamber to the secondary channel while inhibiting the flow of a sample fluid into the secondary channel, wherein each chamber in the plurality of chambers includes an antibiotic with a concentration of the antibiotic dependent on a position of the chamber of said plurality of chambers along the primary channel;
at least one first end opening coupled to the first end of the primary channel and a second end opening coupled to the second end of the primary channel to enable the sample fluid to be loaded into the device either through the at least one first end opening or the second end opening, to flow along the primary channel into the plurality of chambers, and to mix with the antibiotic in each chamber; and
a retaining channel coupled between the primary channel and the secondary channel which allows a retaining fluid in the primary channel to flow into the secondary channel while inhibiting the flow of the sample fluid into the secondary channel so as to isolate droplets of the sample fluid in each chamber of said plurality of chambers.

7. The test kit according to claim 6, wherein the antibiotic comprises an antibiotic fluid.

8. The test kit according to claim 6, wherein the antibiotic comprises a lyophilized antibiotic solute.

9. The test kit according to claim 6, further comprising at least two microstructures on the substrate and a common opening to simultaneously load the sample fluid into the primary channel of the at least two microstructures.

10. The test kit according to claim 6, wherein the retaining fluid comprises FC-40 oil.

11. The test kit according to claim 6, wherein the vent comprises one or a plurality of slits.

12. A method for forming droplets with gradually varied concentrations in a microfluidic device, the method comprising:
in a microstructure formed in a substrate, the microstructure comprising a primary channel with a first end and a second end, and a plurality of chambers that open to the primary channel:
loading through at least two first end openings coupled to the first end of the primary channel, concurrently, at least two fluid streams into the primary channel, which forms, when said at least two fluid streams mix, a fluid mixture having a concentration gradient along the primary channel and the plurality of chambers that are open to that primary channel;
upon loading the plurality of chambers with the fluid mixture, introducing a retaining fluid into the primary channel to purge the fluid mixture from the primary channel while retaining droplets of the fluid mixture in the plurality of chambers—a droplet of said droplets in each of the plurality of chambers, so as to exhibit gradually varied concentrations in the droplets in the plurality of chambers along the primary channel.

13. The method according to claim 12, wherein the retaining fluid comprises a shearing fluid introduced into the first end of the primary channel through a purge opening coupled to the first end so as to purge the fluid of said at least two fluid streams from the primary channel.

14. The method according to claim 12, wherein the shearing fluid comprises air or oil.

15. The method according to claim 12, further comprising computing the concentration of the solute in the droplet using a two-dimensional advection-diffusion equation.

16. The method according to claim 12, wherein loading the at least two fluid streams into the primary channel comprises loading the at least two fluid streams wherein each of the at least two streams include a same antibiotic.

17. The method according to claim 12, wherein loading the at least two fluid streams into the primary channel comprises loading the at least two fluid streams wherein each of the at least two streams include a different antibiotic.

18. The method according to claim 12, wherein the droplets comprise an antibiotic.

19. The method according to claim 18, and further comprising lyophilizing the droplets to form a lyophilized antibiotic solute wherein the mass of the lyophilized antibiotic solute is related to the concentration of the antibiotic in the droplets prior to lyophilization.

20. A method for antibiotic susceptibility testing, the method comprising:
obtaining a antimicrobial susceptibility test (AST) kit, the AST kit comprising:
a microstructure formed in a substrate, the microstructure comprising a primary channel with a first end and a second end, and a plurality of chambers open to the primary channel, each chamber in the plurality of chambers having a volume less than 100 nanoliters and being connected by a vent to a secondary channel in the microstructure, a width of the vent configured to enable a gas to escape from the chamber to the secondary channel while inhibiting the flow of a bacterial sample solution into the secondary channel, wherein each chamber in the plurality of chambers includes an antibiotic with a concentration of the antibiotic dependent on a position of the chamber of said plurality of chambers along the primary channel;
at least one first end opening coupled to the first end of the primary channel and a second end opening coupled to the second end of the primary channel to enable the bacterial sample solution to be loaded into the device either through the at least one first end opening or the second end opening, to flow along the primary channel into the plurality of chambers, and to mix with the antibiotic in each chamber; and
a retaining channel coupled between the primary channel and the secondary channel which allows a retaining fluid in the primary channel to flow into the secondary channel while inhibiting the flow of the bacterial sample solution into the secondary channel so as to isolate droplets of the bacterial sample solution in each chamber of said plurality of chambers;
loading the bacterial sample solution into the primary channel and into the plurality of chambers open to the primary channel allowing the bacterial sample solution to mix with the antibiotic in the droplet in each chamber of the plurality of chambers; and
upon loading the plurality of chambers with the bacterial sample solution, loading the retaining fluid into the primary channel to purge the bacterial sample solution from the primary channel, and into the secondary channel so as to isolate the droplet of the bacterial sample solution with the antibiotic in each chamber of the plurality of chambers.

21. The method for antibiotic susceptibility testing according to claim 20, wherein loading the bacterial sample solution into the primary channel comprises loading the bacterial sample solution through one or more of the at least two first end openings coupled to the first end of the primary channel or through the second opening at the second end of the primary channel.

22. The method for antibiotic susceptibility testing according to claim 20, wherein the antibiotic comprises a lyophilized antibiotic solute.

23. The method for antibiotic susceptibility testing according to claim 20, further comprising:
in an imaging system, monitoring, and acquiring data on, a growth of bacteria in the isolated droplet of bacterial sample solution in each chamber of the plurality of chambers;
in a processor, analyzing the acquired data and computing information about inhibition of the growth of the bacteria based on the antibiotic and concentration of the antibiotic in the isolated droplet in each chamber of the plurality of chambers; and
in an output device, outputting the information.

24. The method for antibiotic susceptibility testing according to claim 23, wherein monitoring the growth of the bacteria comprises using a microscope to image bacterial cells in the isolated droplet in each chamber of the plurality of chambers.

25. The method for antibiotic susceptibility testing according to claim 23, wherein the bacterial sample solution in the droplet comprises a fluorescent indicator, and wherein monitoring the growth of the bacteria comprises analyzing fluorescence from the indicator.

26. The method for antibiotic susceptibility testing according to claim 25, wherein the fluorescent indicator comprises resazurin.

27. The method for antibiotic susceptibility testing according to claim 23, wherein the information comprises a minimal inhibitory concentration (MIC) of the antibiotic.

28. The method for antibiotic susceptibility testing according to claim 23, wherein the information comprises S/I/R determinations about the antibiotic and the bacteria.

29. The method for antibiotic susceptibility testing according to claim 23, wherein monitoring the growth of the bacteria comprises using the imaging system to count the average number of bacteria per chamber of said plurality of chambers.
